(19) United States
(12) Patent Application Publication
(10) Pub. No.: US 2015/0250447 A1
(43) Pub. Date: Sep. 10, 2015

(54) PUNCTURE ADAPTER AND ULTRASOUND PROBE

(71) Applicants: Kabushiki Kaisha Toshiba, Minato-ku (JP); Toshiba Medical Systems Corporation, Otawara-shi (JP)

(72) Inventors: Takashi KUBOTA, Otawara (JP); Takashi TAKEUCHI, Otawara (JP); Yasuhisa MAKITA, Nasushiobara (JP); Minoru AOKI, Yaita (JP)

(73) Assignees: Kabushiki Kaisha Toshiba, Minato-ku (JP); Toshiba Medical Systems Corporation, Otawara-shi (JP)

(21) Appl. No.: 14/720,535

(22) Filed: May 22, 2015

Related U.S. Application Data
(63) Continuation of application No. PCT/JP2013/081490, filed on Nov. 22, 2013.

(30) Foreign Application Priority Data
Nov. 23, 2012 (JP) 2012-256978

(51) Int. Cl. A61B 8/08; A61B 8/12; A61B 8/00; A61B 17/34

(52) U.S. Cl. CPC A61B 8/0841; A61B 17/3403; A61B 8/12; A61B 8/445; A61B 2017/3413

(57) ABSTRACT

A puncture adapter includes a guide of a puncture needle, a gripper to hold an ultrasound probe, and a pinch. The gripper grips the ultrasound probe. The puncture adapter is attached to the ultrasound probe by the engagement between the gripper and the ultrasound probe. The pinch is used to release the grip of the gripper. The pinch is located adjacent to the gripper in a direction that intersects a direction in which the gripper grips the ultrasound probe.

### CROSS-REFERENCE TO RELATED APPLICATION

This application is based upon and claims the benefit of priority from Japanese Patent Application No. 2012-256978, filed 23 Nov. 2012; the entire contents of which are incorporated herein by reference.

### FIELD

Embodiments described herein relate generally to a puncture adapter and an ultrasound probe.

### BACKGROUND

A puncture is a medical procedure usually performed by inserting a puncture needle such as an injection needle in a living body. For example, a puncture is performed for the examination of the tumor by sampling tissues such as cells, the topical administration of a drug, thermal treatment and ablation treatment such as irradiation of microwaves or radio waves from the puncture needle, and the like. With the injection of a drug by puncture, it became possible to treat the case that has required surgery without performing the surgery. The conventional surgical operation may involve significant ablation of tissue, and in some cases the patient has to bear an excessive burden such as the loss of the functions of the tissue and changes in the appearance. In contrast, the puncture can reduce the excessive burden on the patient.

In the puncture, an ultrasound probe is fitted with an instrument (puncture adapter) to guide a puncture needle. The puncture adapter, which is secured to the ultrasound probe, supports the puncture needle and guides it in the insertion direction. That is, the puncture adapter is fixed to the ultrasound probe

The puncture adapter may be fixed to the ultrasound probe in, for example, such a manner as follows. That is, the puncture adapter is fixed by the use of a fixing band that is attached to surround the outer peripheral surface of the ultrasound probe. The fixing band is often configured to be secured by a locking mechanism such as screws. As a result, it may be difficult for the operator to fix a puncture adapter to an ultrasound probe using the fixing band.

For example, while a puncture needle is held by the puncture adapter, the operator grips the ultrasound probe with one hand. At this time, the operator takes a screw of the fixing band in the other hand instead of the puncture needle. The operator then rotates the screw with the other hand to release the locking mechanism of the puncture adapter. In the case of fixing the puncture adapter by using the fixing band with a locking mechanism, operation for fixation or release of the fixation may become complicated. For this reason, there has been used a puncture adapter configured to elastically hold an ultrasound probe.

If elastically holding an ultrasound probe, the puncture adapter has such a configuration as not to shift from the ultrasound probe in the fixed state. For example, a configuration is employed in which a holding part of the puncture adapter is engaged (fitted, etc.) with a held part of the ultrasound probe. In this configuration, one of the parts is convex, and the other is concave. In the holding state, the convex part fits in the concave part, and thus the fixed state of the ultrasound probe and the puncture adapter is retained.

When the operator removes the puncture adapter from the ultrasound probe, the holding state of the puncture adapter is released. The release of the holding state requires to release the engagement between the holding part of the puncture adapter and the held part of the ultrasound probe. To release the engagement, the operator separates (opens) holding parts of the puncture adapter from each other against elasticity to release the ultrasound probe. For example, the operator removes the convex part from the concave part.

The puncture adapter is provided with a functional part related to the removal operation from the ultrasound probe. This functional part is intended to release the engagement as described above, and includes, for example, a pair of operation parts for separating the holding parts. For example, as in a common pinch, the holding parts separate from each other when the operation parts are brought close to each other against elasticity.

In such a configuration, the greater required force is to separate the holding parts from each other, the operability of the removal operation of the puncture adapter decreases. Further, if the operator removes the puncture adapter while the holding state remains not completely released, attached substance (blood, etc.) on the puncture adapter or the ultrasound probe may be scattered.

On the other hand, by increasing the distance between the operation parts and the holding parts of the puncture adapter, the operability of the removal operation may be improved. However, it is necessary to avoid that the operation parts protrude significantly in a direction toward the outer peripheral surface from the central axis of the ultrasound probe. For example, if the operation parts protrude significantly from the outer peripheral surface of the ultrasound probe, which may interfere with the puncture. In addition, if the puncture adapter is to be attached to an intracavitary probe or the like that is inserted in the object, the burden on the insertion part may increase as the amount of protrusion from the outer peripheral surface increases in the operation parts.

However, if the puncture adapter is designed to be small not to interfere with the puncture and to reduce the burden, it is difficult to secure the distance between the operation parts and the holding parts. This increases the rigidity of a connection part. Accordingly, a larger operating force (a force to grip the pair of the operation parts) is required for the removal operation, which makes the removal difficult.

### BRIEF DESCRIPTION OF THE DRAWINGS

FIG. 1 is a schematic perspective view of an ultrasound probe according to a first embodiment;

FIG. 2 is a schematic perspective view of a puncture adapter of the first embodiment;

FIG. 3 is a schematic perspective view of the main body of the puncture adapter of the first embodiment;

FIG. 4 is a schematic perspective view of a cover of the puncture adapter of the first embodiment;

FIG. 5 is a schematic perspective view of the puncture adapter illustrated in FIG. 2 viewed from the back;

FIG. 6 is a schematic partial enlarged view of a gripper and a first operation part of the adapter body illustrated in FIG. 5;

FIG. 7 is a schematic perspective view of an ultrasound probe fitted with the puncture adapter of the first embodiment;

FIG. 8 is a schematic cross-sectional view taken along line A-A in FIG. 7;

FIG. 9 is a schematic cross-sectional view taken along line B-B in FIG. 7;

FIG. 10 is a schematic cross-sectional view taken along line C-C in FIG. 7;

FIG. 11A is a schematic cross-sectional view conceptually illustrating the engaged state of the gripper of the adapter body of the first embodiment;

FIG. 11B is a schematic cross-sectional view conceptually illustrating the non-engaged state of the gripper of the adapter body of the first embodiment;

FIG. 12 is a schematic cross-sectional view of a probe cover of the ultrasound probe and engaging parts of the puncture adapter;

FIG. 13A is a schematic perspective view of an example of an ultrasound probe according to a second embodiment;

FIG. 13B is a schematic view of a proximal end side engaging part, a sub engaging part, and a slope of the ultrasound probe of the second embodiment;

FIG. 14A is a schematic cross-sectional view of the proximal end side engaging part and the like of the ultrasound probe of the second embodiment;

FIG. 14B is a schematic cross-sectional view of the proximal end side engaging part and the like of the ultrasound probe of the second embodiment; and

FIG. 14C is a schematic cross-sectional view of the proximal end side engaging part and the like of the ultrasound probe of the second embodiment.

### DETAILED DESCRIPTION

In general, according to one embodiment, a puncture adapter includes a guide of a puncture needle, a gripper to grip an ultrasound probe, and a pinch. The gripper holds the ultrasound probe. The puncture adapter is attached to the ultrasound probe by the engagement between the gripper and the ultrasound probe. The pinch is used to release the grip of the gripper. The pinch is located adjacent to the gripper in a direction that intersects a direction in which the gripper grips the ultrasound probe.

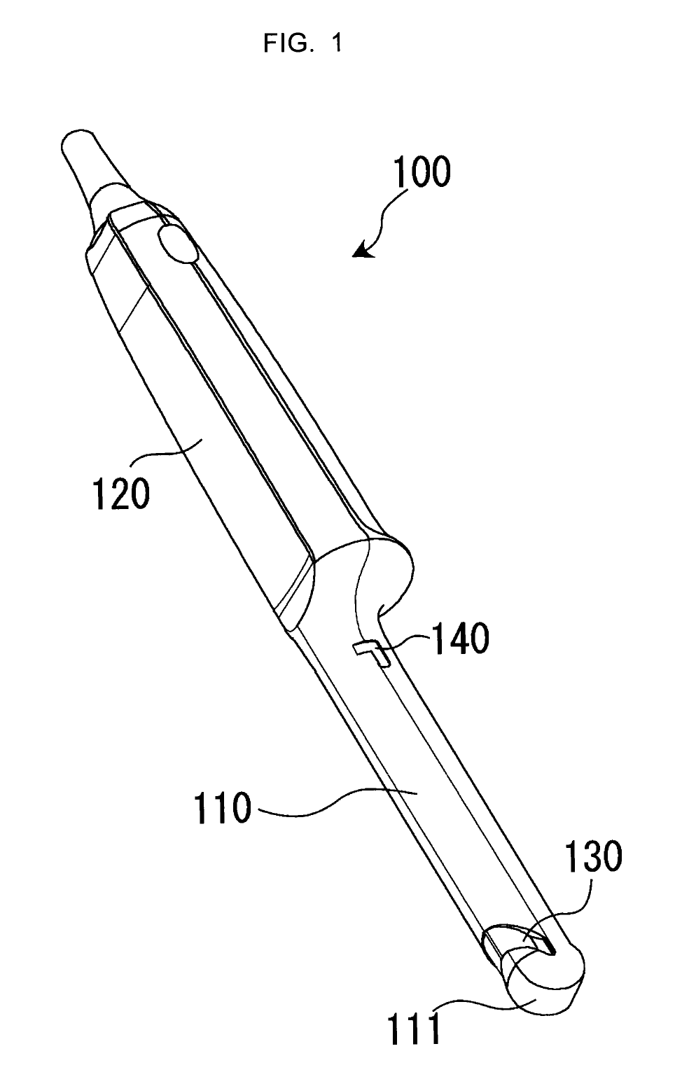

In the following, a description is given of an example of a puncture adapter and an ultrasound probe (ultrasound probe body) according to embodiments with reference to FIGS. 1 to 14C. The puncture adapter of the embodiments is attachable/detachable to/from the ultrasound probe. While the puncture adapter of the embodiments is described below as being attached to an intracavitary ultrasound probe that is insertable in a subject, the ultrasound probe is not limited to be of a predetermined shape.

### First Embodiment

### Configuration of the Ultrasound Probe

A description is given of the configuration of an ultrasound probe according to a first embodiment. FIG. 1 is a schematic perspective view of an ultrasound probe 100 of the embodiment. FIG. 1 illustrates the ultrasound probe 100 without a puncture adapter 200. As illustrated in FIG. 1, the ultrasound probe 100 includes an insertion part 110 to be inserted into a subject, and a handle 120 that the user grips. In the example of FIG. 1, the insertion part 110 has a columnar shape with a curved end. The proximal end side of the insertion part 110 forms a boundary with the handle 120. The outer peripheral surface of the insertion part 110 is continuous with that of the handle 120.

The distal end of the insertion part 110 accommodates an ultrasound generator 111 in the ultrasound probe 100. That is, a plurality of ultrasound transducers are provided as the ultrasound generator 111 inside the distal end of the insertion part 110. When the ultrasound probe 100 receives a drive signal from an ultrasound diagnostic apparatus, the ultrasound transducers are driven, and thereby the ultrasound generator 111 generates ultrasound waves. The ultrasound transducers also receive reflected waves from a subject. An acoustic lens or the like is provided on the distal end surface of the ultrasound probe 100 (the insertion part 110). The ultrasound transducers may form a one-dimensional array in which they are arrayed in a scanning direction or two-dimensional arrays.

Although not illustrated, the handle 120 is connected to a cable for transmitting/receiving signals to/from the ultrasound diagnostic apparatus. In addition, the handle 120 may be provided with an electrical circuit for processing signals fed from the ultrasound diagnostic apparatus and the ultrasound transducers.

The ultrasound probe 100 transmits ultrasound waves to the subject based on a signal received from the ultrasound diagnostic apparatus. The ultrasound probe 100 converts the reflected waves from the subject into an electrical signal (echo signal) by the ultrasound transducers, and sends the electrical signal to the ultrasound diagnostic apparatus. The ultrasound diagnostic apparatus visualizes the biological information (morphological information, functional information, etc.) of the subject into an image based on the electrical signal received from the ultrasound probe 100. Such electrical processing and image processing are performed in a conventional manner.

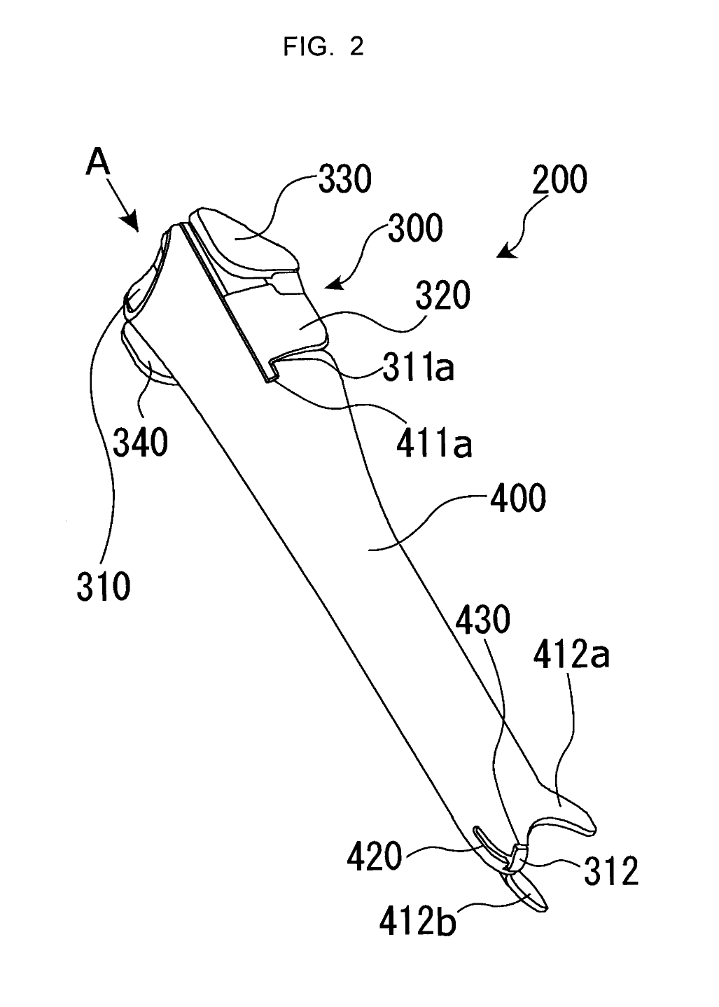

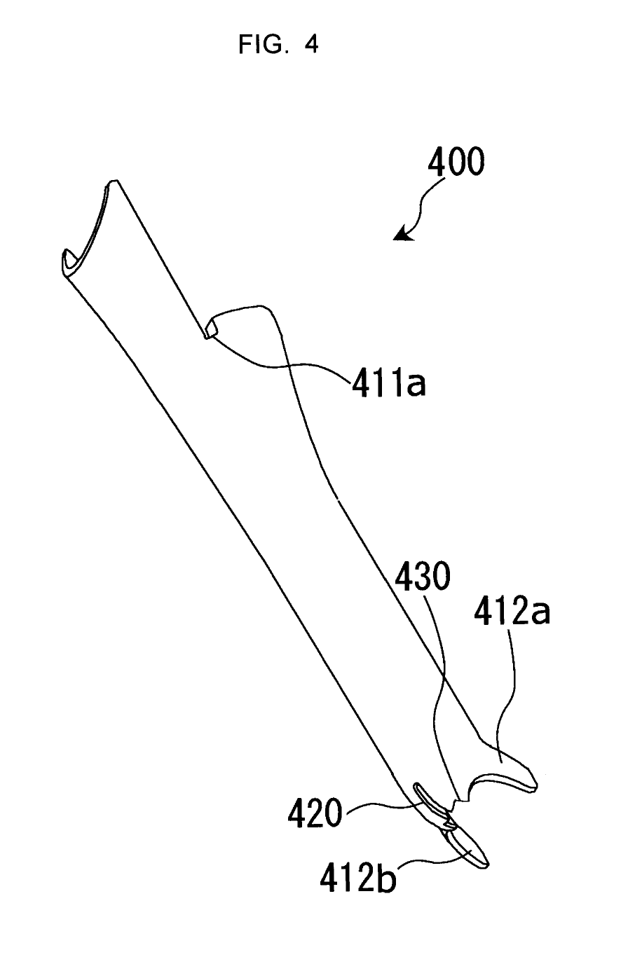

The ultrasound probe 100 is provided with a distal end side engaging part 130 and a proximal end side engaging part 140 on its outer peripheral surface. In the example of FIG. 1, the distal end side engaging part 130 is located in the vicinity of the ultrasound generator 111 in the insertion part 110, and is formed in a recess that engages each of engaging protrusions 412a and 412b (described later) of the puncture adapter 200 (see FIGS. 2 and 4). A pair of the distal end side engaging parts 130 is arranged on the distal end side of the insertion part 110 so that they are interposed between the engaging protrusions 412a and 412b of the puncture adapter 200. Also in the example of FIG. 1, the proximal end side engaging part 140 is located in the insertion part 110 in the vicinity of the boundary with the handle 120, and is formed in a recess that engages a gripper 320 (see FIG. 2) of the puncture adapter 200.

### [Configuration of the Puncture Adapter]

The puncture adapter 200 is described below with reference to FIGS. 2 to 11B. The puncture adapter 200 is attached and fixed to the ultrasound probe 100 by engagement. The puncture adapter 200 has a function of guiding a puncture needle in the insertion direction when the puncture needle is inserted therein.

### (Outline of the Puncture Adapter 200)

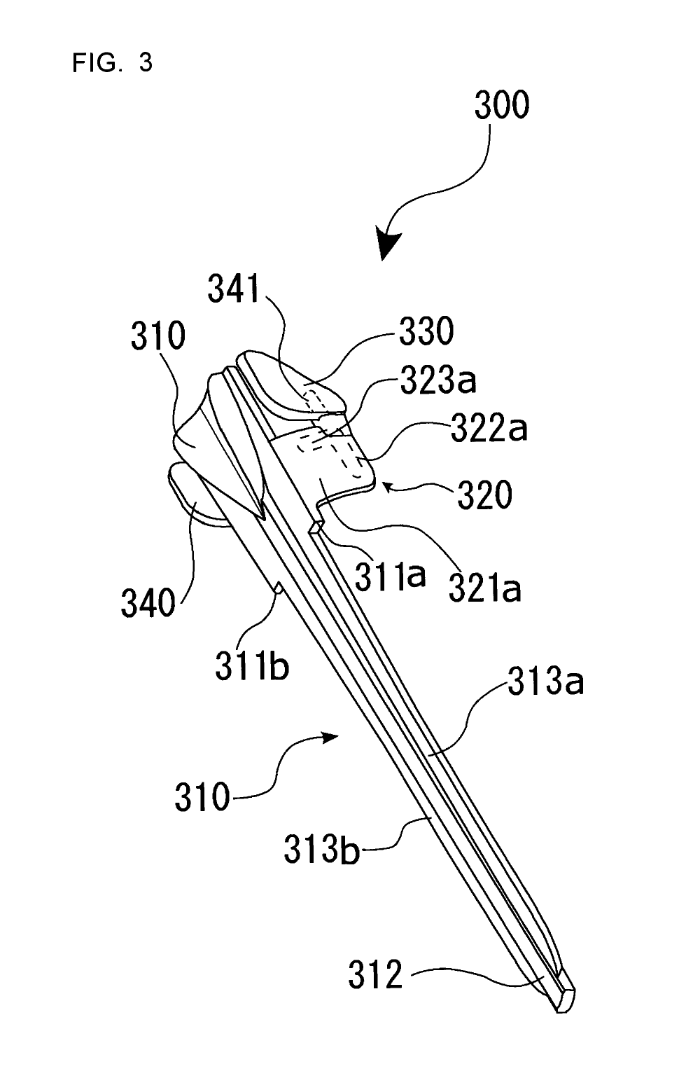

The outline of the puncture adapter 200 is described with reference to FIGS. 2 to 4. FIG. 2 is a schematic external view of the puncture adapter 200 of the first embodiment. FIG. 3 is a schematic external view of an adapter body 300 of the puncture adapter 200 of the first embodiment. FIG. 4 is a schematic external view of a cover 400 of the puncture adapter 200 of the first embodiment. As illustrated in FIGS. 2 to 4, the puncture adapter 200 includes the adapter body 300 and the cover 400. The puncture adapter 200 further includes a plurality of engaging parts each for engaging one of the distal end side engaging parts 130 and the proximal end side engaging parts 140 of the ultrasound probe 100. The puncture adapter 200 is attached to the ultrasound probe 100 through the engaging parts. As illustrated in FIGS. 2 and 4, the cover 400 of the puncture adapter 200 is configured to be detachably attached to the adapter body 300.

While the puncture adapter 200 is configured to be separated into the adapter body 300 and the cover 400, it may be formed integrally. However, in the first embodiment, the puncture adapter 200 can be separated into the adapter body 300 and the cover 400 to improve the maintenance efficiency such as facilitating the cleaning after the removal of the puncture adapter 200 from the ultrasound probe 100.

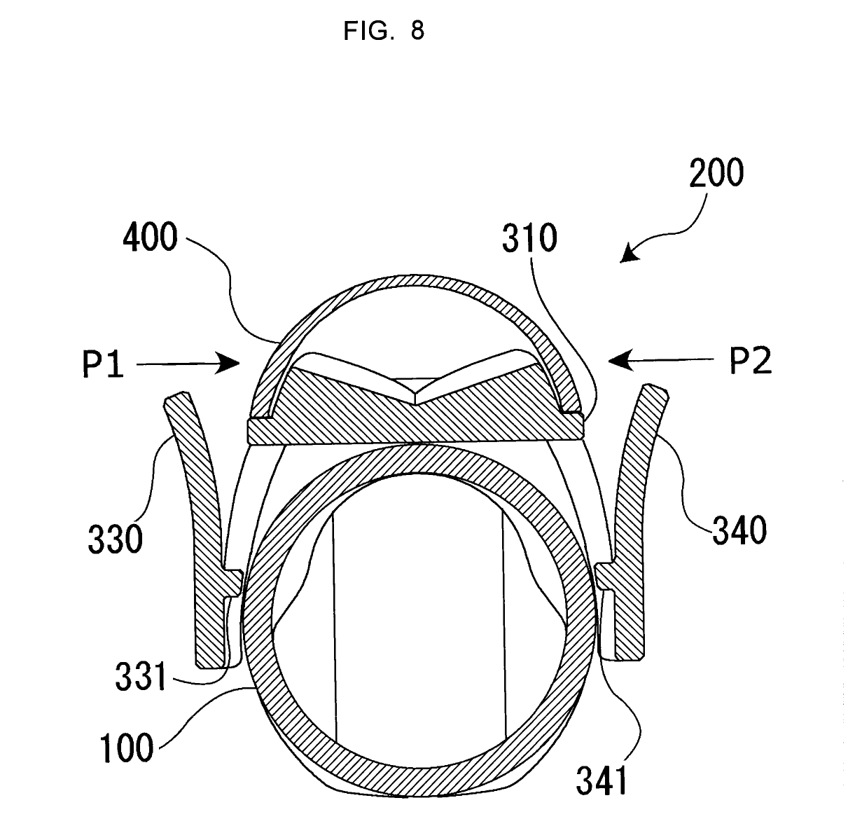

A puncture needle (not illustrated) is inserted through a space formed between a guide plate 312 of a plate-like member 310 of the adapter body 300 (see FIG. 3) and the inner peripheral surface of the cover 400 (see FIG. 8). The puncture needle is guided in the insertion direction by the guide plate 312 and the inner peripheral surface (see arrow A in FIG. 2). This insertion direction is a direction that intersects with the cross section of FIG. 8 (perpendicular direction, etc.). Incidentally, at least one of the guide plate 312 and the inner peripheral surface corresponds to an example of “guide”.

### (Configuration of the Adapter Body 300)

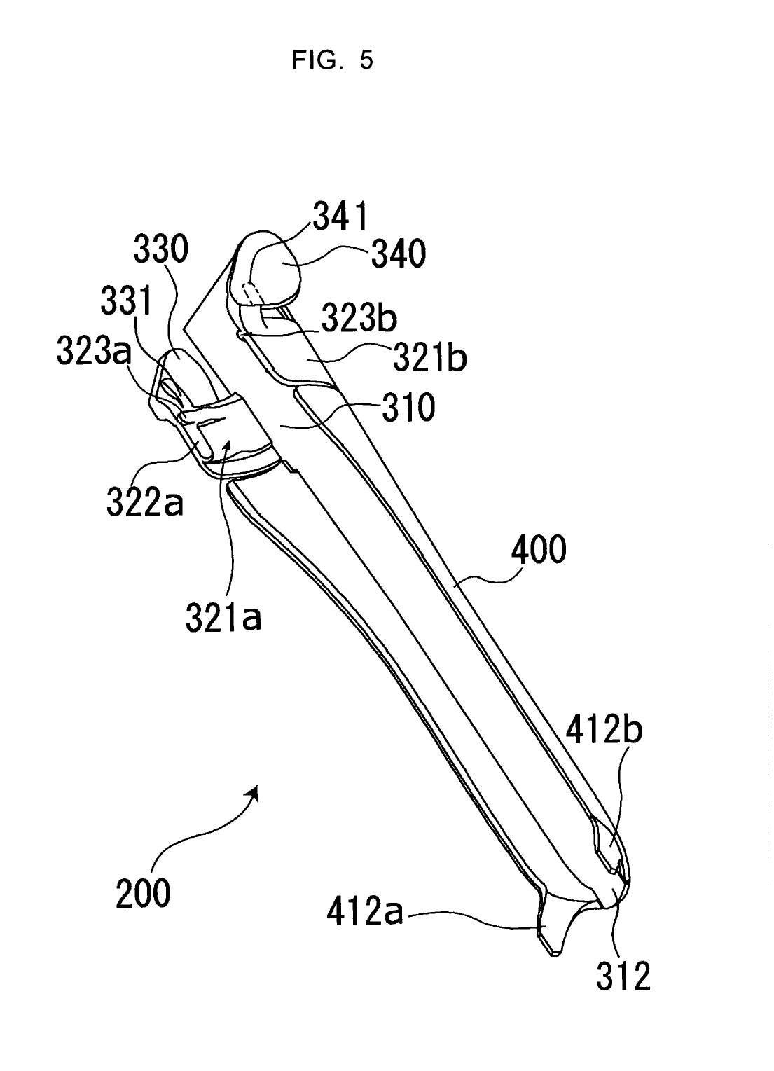

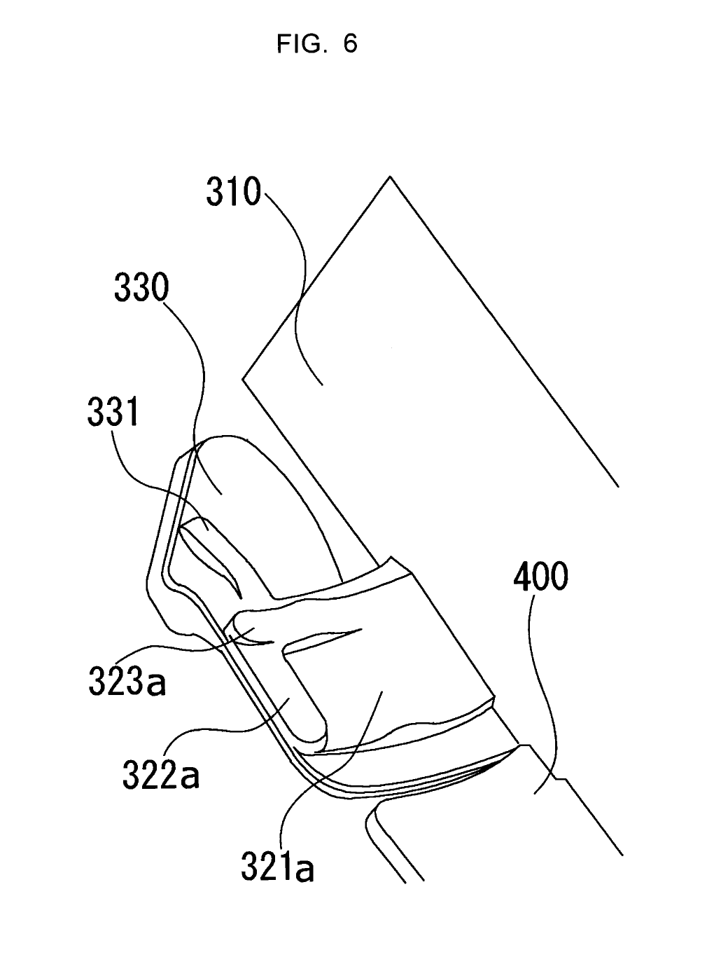

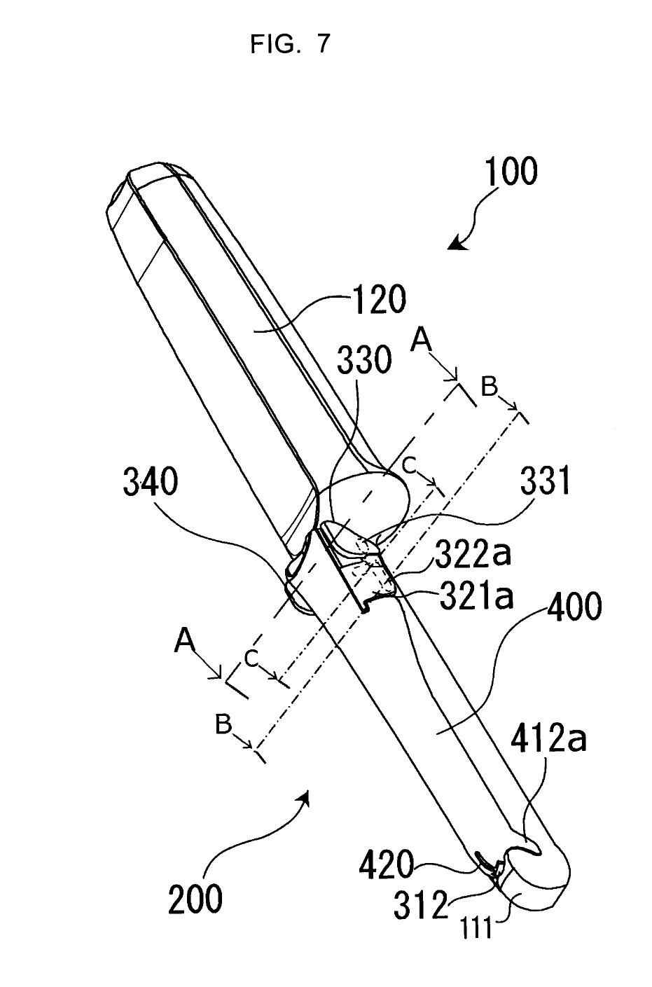

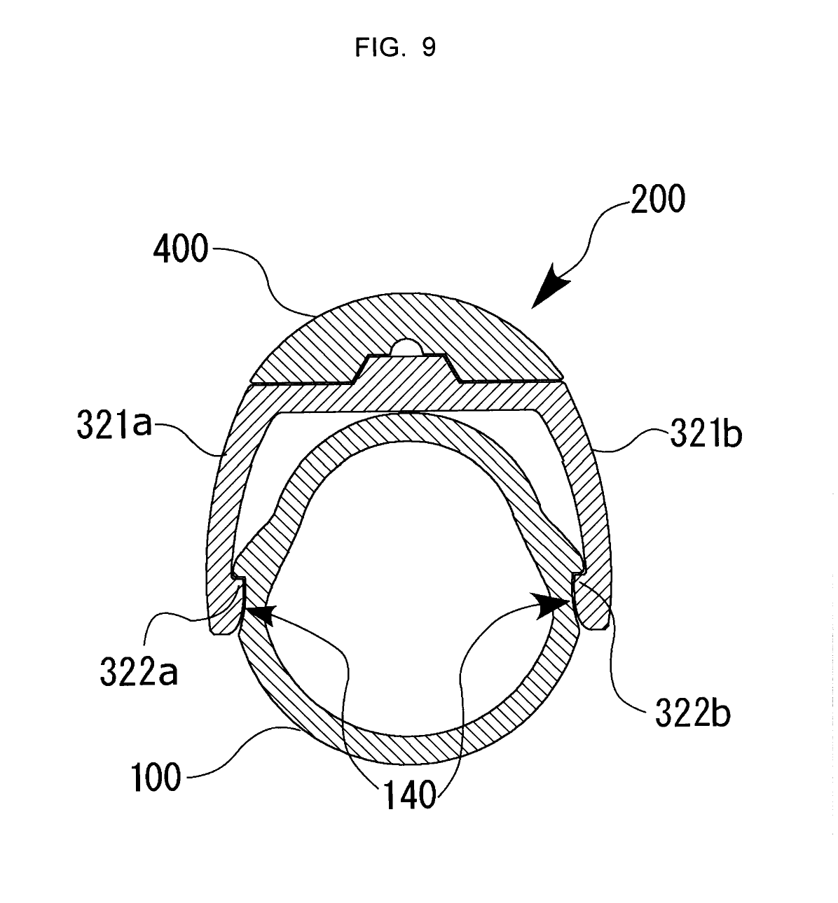

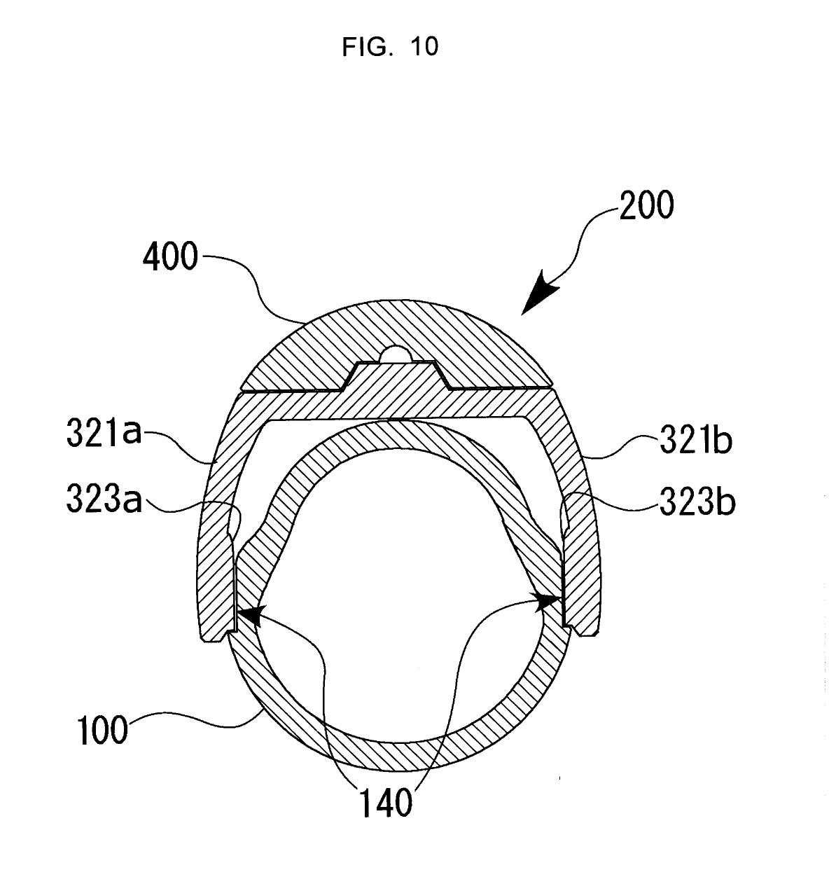

Next, the configuration of the adapter body 300 is described with reference to FIGS. 2 to 10. FIG. 5 is a schematic perspective view of the puncture adapter 200 illustrated in FIG. 2 viewed from the back. FIG. 6 is a schematic partial enlarged view of the gripper 320 and a first operation part 330 of the adapter body 300 illustrated in FIG. 5. FIG. 7 is a schematic perspective view of the ultrasound probe 100 fitted with the puncture adapter 200 of the first embodiment. FIGS. 8, 9 and 10 are schematic cross-sectional views respectively taken along lines A-A, B-B and C-C in FIG. 7. FIG. 8 illustrates the first operation part 330 and a fulcrum 331, and a second operation part 340 and a fulcrum 341. FIG. 9 illustrates a first holding part 321a and a first engaging part 322a, and a second holding part 321b and a second engaging part 322b. FIG. 10 illustrates the first holding part 321a and a first engaging part 323a, and the second holding part 321b and a second engaging part 323b.

Note that the first operation part 330 and the second operation part 340 correspond to an example of “pinch”.

### <Overall Configuration>

As illustrated in FIG. 3, the adapter body 300 includes the plate-like member 310, the gripper 320, the first operation part 330, and the second operation part 340. The plate-like member 310 is a flat plate, and gradually becomes narrower from the proximal end toward the distal end. In the example of FIG. 3, the plate-like member 310 has a portion having a substantially constant first width at the proximal end. The first width continues to some extent toward the distal end side (in the puncture direction), and in the middle of the way to the distal end, the first width reduces to a second width. The plate-like member 310 gradually becomes further narrower toward the distal end from where the first width changes to the second width. FIG. 3 illustrates a boundary at which the first width changes to the second width. That is, at the boundary where the width changes, steps 311a and 311b are formed in the width direction on the right and left of the plate-like member 310, respectively. The step 311a is fitted to a fitting part 411a of the cover 400 (described later). Similarly, the step 311b is fitted to a fitting part of the cover 400. With this, the adapter body 300 and the cover 400 are combined and fixed.

### <Plate-Like Member>

The plate-like member 310 includes the guide plate 312, with which the puncture needle makes contact in the longitudinal direction from the proximal end toward the distal end, and side plates 313a and 313b that protrude substantially parallel to the guide plate 312 on the right and left of the guide plates 312, respectively, and extend along the longitudinal direction of the guide plates 312. The side plates 313a and 313b are each configured to abut on the inner peripheral surface of the cover 400, thereby reducing the misalignment between the adapter body 300 and the cover 400.

### <Overall Configuration of the Gripper>

Next, with reference to FIGS. 3, and 5 to 10, the overall configuration of the gripper 320 is described. As illustrated in FIGS. 3 and 5, the adapter body 300 is provided with the gripper 320 having a curved surface along the outer peripheral surface of the ultrasound probe 100. The gripper 320 protrudes from both side edges of the plate-like member 310 in a direction away from the plate-like member 310. As illustrated in the figures, the gripper 320 is located on the proximal end side of the step 311a of the plate-like member 310. The gripper 320 includes the first holding part 321a, the second holding part 321b, and a part of the plate-like member 310 as their connector. That is, the first holding part 321a protrudes from one side edge of the plate-like member 310 toward one of the proximal end side engaging parts 140 on the outer peripheral surface of the ultrasound probe 100 at the time of attachment. The second holding part 321b protrudes from the other side edge toward the other of the proximal end side engaging parts 140 on the outer peripheral surface of the ultrasound probe 100. The plate-like member 310 located between the first holding part 321a and the second holding part 321b has the function of a connector for connecting them. The first holding part 321a and the second holding part 321b face across the connector. That is, the first holding part 321a, the connector, and the second holding part 321b forms the gripper 320 together in a shape that surrounds the outer peripheral surface of the ultrasound probe 100.

### <Configuration of Holding Part>

With reference to FIGS. 5 and 6, the first holding part 321a and the second holding part 321b are described below. Note that the first holding part 321a and the second holding part 321b are bilaterally symmetrical, and thus but one of them, i.e., the first holding part 321a is described to explain their configuration. The description of the second holding part 321b is omitted.

As illustrated in FIGS. 5 and 6, the first engaging part 322a is arranged in the vicinity of the distal end of the first holding part 321a that protrudes from the side edge of the plate-like member 310 in a direction away therefrom. The first engaging part 322a extends in a direction perpendicular to the protruding direction. The first engaging part 322a protrudes or is raised toward the second engaging part 322b that faces the inner surface of the first holding part 321a. The first engaging part 322a has a shape corresponding to the recess of the proximal end side engaging part 140 in the insertion part 110 of the ultrasound probe 100 (see FIG. 1), and is engaged with the proximal end side engaging part 140 when the gripper 320 is in the closed state (see FIGS. 9 and 10). Note that the closed state refers to a state of the gripper 320 when the removal operation or the like is not performed by the operator. In the closed state, i.e., when the operator is not applying a predetermined pressing force to the first operation part 330 and the second operation part 340, the distance between the first engaging part 322a and the second engaging part 322b is the shortest.

Besides, as illustrated in FIGS. 5 and 6, the first holding part 321a is provided with the first engaging part 323a that is formed to be continuous with one longitudinal end of the first engaging part 322a. As with the first engaging part 322a, the first engaging part 323a protrudes or is raised from the inner surface of the first holding part 321a toward the second engaging part 322b that faces the first holding part 321a. The first engaging part 323a is arranged to have a predetermined length from the proximal end side (the plate-like member 310 side) of the first holding part 321a in a direction away from the first engaging part 322a. As illustrated in FIG. 6, the first engaging part 323a has a shape corresponding to the recess of the proximal end side engaging part 140 in the insertion part 110 of the ultrasound probe 100 (see FIG. 1), and is engaged with the proximal end side engaging part 140 when the gripper 320 is in the closed state (see FIGS. 9 and 10).

One end of the first engaging part 322a is formed to be continuous with one end of the first engaging part 323a. The continuous protrusions are engaged with the proximal end side engaging part 140. In the example of FIG. 6, the combination of the first engaging parts 322a and 323a forms a substantially L-shape. In the above description referring to examples of FIGS. 5 and 6, the first engaging part 322a is distinguished from the first engaging part 323a for convenience of description. The first engaging parts (322a, 323a) form a protrusion to be engaged with the proximal end side engaging part 140. However, this is not so limited, and the first engaging parts 322a and 323a may be separated from each other, or there may be three or more independent protrusions. If the proximal end side engaging parts 140 are convex, the first engaging parts 322a and 323a are formed in a concave shape. If the proximal end side engaging parts 140 are a combination of convex and concave parts, the first engaging part 322a and the like are formed to be a combination of concave and convex parts corresponding thereto. The same applies to the second engaging parts 322b and 323b described below.

As described above, the second holding part 321b is bilaterally symmetrical to the first holding part 321a, and the combination of the second engaging parts 322b and 323b has a substantially L-shape symmetrical to the L-shape described above. As with the first engaging parts 322a and 323a, the second engaging parts 322b and 323b are formed so that their shape corresponds to the recess of the other proximal end side engaging part 140 of the insertion part 110. The second engaging parts 322b and 323b are engaged with the proximal end side engaging part 140 when the gripper 320 is in the closed state. The first holding part 321a and the second holding part 321b have elasticity as well as flexibility. Therefore, when the first holding part 321a and the second holding part 321b are opened by the operator in the direction away from each other, the elasticity causes a force that brings them to return to their original positions. Accordingly, the first holding part 321a and the second holding part 321b are biased in a direction in which they come close to each other. By the biasing force of the gripper 320, the gripper 320 holds the insertion part 110 of the ultrasound probe 100.

<Configuration of the First Operation Part and the Second Operation Part>

Next, with reference to FIGS. 3 and 5 to 8, the configuration of the first operation part 330 and the second operation part 340 are described. As illustrated in FIG. 5, the first operation part 330 and the second operation part 340 are arranged to face each other across the plate-like member 310. The first operation part 330 and the second operation part 340 have elasticity as well as flexibility. When the operator applies a predetermined pressing force to the operation parts, the first operation part 330 and the second operation part 340 come close to each other. On the other hand, if the operator lowers the pressure on the operation parts or removes his/her hand therefrom, the first engaging part 322a and the second engaging part 322b return to their original positions by the elastic force of the gripper 320 and the like. In addition, by the elastic force of the gripper 320, the first operation part 330 and the second operation part 340 are biased in a direction in which they are separated from each other. In other words, the first engaging parts (322a, 323a) and the second engaging parts (322b, 323b) are each configured to move by the biasing force to the original position from a position when the operation parts are being pressed.

As illustrated in FIGS. 3 and 5, the adapter body 300 is provided with the first operation part 330 that is adjacent to the first holding part 321a in the proximal end direction of the plate-like member 310 and is connected to the first holding part 321a. In the example of FIG. 6, the first holding part 321a and the first operation part 330 are formed continuously. As illustrated in FIG. 5, the first operation part 330 is connected to the first holding part 321a on an extension of the first engaging part 322a in the extending direction, and other parts are disconnected. In other words, a part of the first operation part 330 that is pressed (effort), the fulcrum 331 of the first operation part 330, and the first engaging part 322a (operating point) are not aligned in a straight line. That is, the first holding part 321a and the first operation part 330 are connected such that the effort and the operating point are misaligned upon opening and closing the gripper 320.

The proximal end direction of the plate-like member 310 with respect to the first holding part 321a corresponds to an example of “a direction that intersects a direction in which the gripper grips the ultrasound probe”.

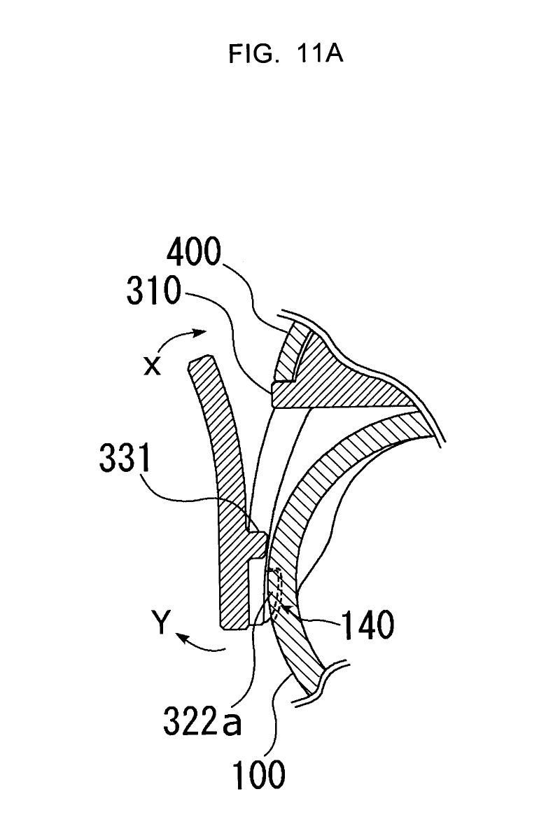

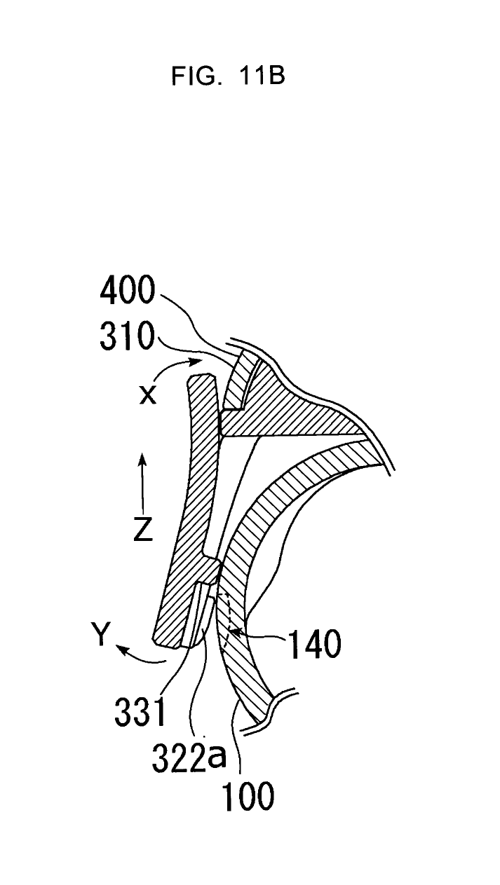

In addition, as illustrated in FIG. 6, the fulcrum 331 that protrudes toward the second operation part 340 is arranged on the surface of the first operation part 330 facing the second operation part 340. When attaching the puncture adapter 200 to the ultrasound probe 100, the operator presses the first operation part 330 against the outer peripheral surface of the insertion part 110 (see direction P1 in FIG. 8). With this, the fulcrum 331 abuts on the outer peripheral surface of the ultrasound probe 100 (see FIGS. 11A and 11B). As a result, the force that presses the first operation part 330 against the outer peripheral surface of the ultrasound probe 100 acts to bring the first holding part 321a away from the outer peripheral surface through the fulcrum 331. For more details, the function of the puncture adapter 200 is described later with reference to FIGS. 11A and 11B.

As described above, the first operation part 330 is adjacent to the first holding part 321a on the proximal end side of the plate-like member 310 (see FIG. 5). Accordingly, while the puncture adapter 200 is being attached to the ultrasound probe 100, when strongly pressed against the outer peripheral surface of the insertion part 110 (see direction P1 in FIG. 8), the first operation part 330 makes contact with the outer peripheral surface of the plate-like member 310 or the cover 400. That is, the puncture adapter 200 is configured to limit the movable range of the first operation part 330. Thus, even if excessive pressing force is applied to the first operation part 330, it is possible to prevent damage to the adapter body 300 including the first operation part 330.

The second operation part 340 is bilaterally symmetrical to the first operation part 330. Therefore, the connection relationship between the second operation part 340 and the second holding part 321b is similar to that between the first operation part 330 and the first holding part 321a. That is, the second operation part 340 is adjacent to the second holding part 321b in the proximal end direction of the plate-like member 310. The second operation part 340 is connected to the second holding part 321b. The fulcrum 341 that protrudes toward the first operation part 330 is arranged on the surfaces of the second operation part 340 facing the first operation part 330. Similar to the fulcrum 331, when the second operation part 340 is pressed against the outer peripheral surface of the insertion part 110 (see direction P2 in FIG. 8), the fulcrum 341 abuts on the outer peripheral surface of the ultrasound probe 100 (see FIGS. 11A and 11B). With this, the force that presses the second operation part 340 acts in the direction of releasing the engagement of the first holding part 321a. For more details, the function of the puncture adapter 200 is described later with reference to FIGS. 11A and 11B.

Also, as with the first operation part 330, when strongly pressed against the outer peripheral surface of the insertion part 110 (see direction P2 in FIG. 8), the second operation part 340 makes contact with the outer peripheral surface of the plate-like member 310 or the cover 400. That is, the puncture adapter 200 is configured to limit the movable range of the second operation part 340. Thus, even if excessive pressing force is applied to the second operation part 340, it is possible to prevent damage to the adapter body 300 including the second operation part 340.

The first operation part 330 and the second operation part 340 each have a curved shape at the edge on the distal end side of the plate-like member 310. This shape is further described. The embodiment has described the puncture adapter 200 that is attached to an intracavitary probe as an example. In the puncture adapter 200, the first operation part 330 and the second operation part 340 are located on the proximal end side of the insertion part 110. This is because the size around the operation parts tends to be large due to the operation parts that are operated as being pinched.

In the puncture adapter 200 that is attached to the ultrasound probe 100, the size around the operation parts includes the size of the ultrasound probe 100 having a predetermined diameter (or width/thickness) and the protruding length of the operation parts surrounding it. The operation parts are likely to be formed in protrusions to reduce the force required for the pinch operation. Accordingly, the size may become thicker radially from the central axis of the ultrasound probe 100. For example, the operation parts (330, 340) of the puncture adapter 200 are located in the vicinity of the boundary between the insertion part 110 and the handle 120 so that they are spaced apart as much as possible from the distal end of the insertion part 110 of the probe to be inserted into the human body. In the configuration in which the operation parts are located in such positions, the smooth curved surface of the distal end edge of each operation part has advantages that, when the operation part makes contact with the insertion opening as the insertion length of the insertion part 110 increases, less burden is placed on the insertion opening of the subject.

Note that the first operation part 330 and the second operation part 340 need not be directly connected to the first holding part 321a and the second holding part 321b, respectively. Any configuration may be employed as long as the first holding part 321a and the second holding part 321b separate from each other in response to pressing of the first operation part 330 and the second operation part 340. For example, the first operation part 330 and the second operation part 340 may be configured to abut on the first holding part 321a and the second holding part 321b, respectively, to transmit a pressing force applied thereto to the holding parts. Besides, the locations of the fulcrums 331 and 341 of the first operation part 330 and the second operation part 340 are not limited to the positions illustrated in FIG. 6. These positions are on the facing surfaces of the operation parts. The fulcrum 331 is located between the first engaging part 322a and a part of the first operation part 330 which is pressed. Similarly, the fulcrum 341 is located between the second engaging part 322b and a part of the second operation part 340 which is pressed. The protruding shape of the fulcrums 331 and 341 is not limited to a linear shape as illustrated in FIG. 6, and may be a point-like shape. Also, the shape of each operation part in the plane direction is not limited to a linear shape as illustrated in FIG. 6, and may be a point-like shape.

Incidentally, the fulcrums 331 and 341 correspond to examples of “first protrusion” and “second protrusion”, respectively.

### (Cover 400)

Next, with reference to FIGS. 4 and 8, the configuration of the cover 400 is described. As illustrated in FIGS. 4 and 8, the cover 400 has a generally semi-cylindrical shape. That is, the cover 400 has an arcuate curved surface. In addition, the cover 400 has a predetermined length in the longitudinal direction perpendicular to the curved surface. As illustrated in FIG. 4, the cover 400 is formed wider in positions corresponding to the operation parts and the gripper 320 of the adapter body 300, and gradually becomes narrower toward the distal end side. Further, the cover 400 is provided with the fitting part 411a for receiving the step 311a of the adapter body 300 at a position corresponding to the step 311a. Similarly, the cover 400 is provided with a fitting part (not illustrated) for receiving the step 311b at a position corresponding to the step 311b. When the steps 311a and 311b are each fitted in the fitting part 411a or the like (see FIG. 2), the cover 400 is attached to the adapter body 300 and fixed.

In addition, as illustrated in FIGS. 2 and 4, the cover 400 is provided, on the distal end side, with the engaging protrusions 412a and 412b corresponding to the insertion part 110 and the pair of the distal end side engaging parts 130 of the ultrasound probe 100. Since the distal end side engaging part 130 is a recess as illustrated in each figure, the engaging protrusions 412a and 412b are each formed in a protruding shape corresponding to the recess. Each of the distal end side engaging parts 130 engages one of the engaging protrusions 412a and 412b, and thereby the distal end side of the puncture adapter 200 and the distal end side of the insertion part 110 of the ultrasound probe 100 are fixed.

Also, as illustrated in FIGS. 2 and 4, the cover 400 is provided with a puncture needle outlet 420 at a position between the engaging protrusions 412a and 412b on the distal end side. The cover 400 is further provided with a guide plate engaging part 430 for receiving the distal end of the guide plate 312 at a position slightly below the distal end of the puncture needle outlet 420 (on the ultrasound probe 100 side). The guide plate engaging part 430 is formed in a recess and receives the distal end of the guide plate 312, thereby achieving the positioning of the distal end of the guide plate 312. Further, by the engagement between the guide plate engaging part 430 and the guide plate 312, the adapter body 300 and the cover 400 are fixed on the distal end side of the puncture adapter 200.

As illustrated in FIG. 2, the puncture needle is inserted from a puncture needle insertion opening between the proximal end of the plate-like member 310 and the proximal end of the cover 400 (see upper part in FIG. 8). On the distal end side, the puncture needle comes out from the puncture needle outlet 420 between the cover 400 and the guide plate 312.

### [Action of the Puncture Adapter]

In the following, with reference to FIGS. 8 to 11B, a description is given of the pinch operation on the first operation part 330 and the second operation part 340, and the action of the gripper 320 corresponding to the operation. FIG. 11A is a schematic cross-sectional view conceptually illustrating the engaged state of the gripper 320 of the adapter body 300 of the embodiment. FIG. 11B is a schematic cross-sectional view conceptually illustrating the non-engaged state of the gripper 320 of the adapter body 300 of the embodiment. FIG. 11A illustrates a state of the operation part before receiving the pressing operation. FIG. 11B illustrates a state of the operation part being pressed.

As illustrated in FIG. 9, before the first operation part 330 and the second operation part 340 receive the pinch operation by the operator, the first engaging part 322a and the second engaging part 322b are each engaged with one of the proximal end side engaging parts 140.

As indicated by directions P1 and P2 in FIG. 8, when the first operation part 330 and the second operation part 340 are pressed to approach each other by the operator, as illustrated in FIG. 11A, each operation part comes close to the ultrasound probe 100 (X direction). At this time, the fulcrums 331 and 341 are brought into contact with the outer peripheral surface of the ultrasound probe 100. Thus, by using leverage where the first operation part 330 is the effort, the fulcrum 331 is the fulcrum, and the first engaging part 322a is the operating point, the first engaging parts 322a and 323a of the embodiment move in a direction (Y direction) away from the outer peripheral surface of the ultrasound probe 100. Similarly, by using the second operation part 340 as the effort, the fulcrum 341 as the fulcrum, and the second engaging part 322b as the operating point, the second engaging parts 322b and 323b move in a direction (Y direction) away from the outer peripheral surface of the ultrasound probe 100.

Thereby, as illustrated in FIG. 11B, the engagement between the proximal end side engaging parts 140 and the first engaging part 322a and the second engaging part 322b is released. Thus, the puncture adapter 200 can be removed from the ultrasound probe 100. Further, as illustrated in FIG. 11B, even in the released state, the fulcrums 331 and 341 are in contact with the outer peripheral surface of the ultrasound probe 100. Since the ultrasound probe 100 has a curved outer peripheral surface, the fulcrums 331 and 341 slid along the outer peripheral surface of the ultrasound probe 100 (Z direction), and act to facilitate the removal of the puncture adapter 200. In this regard, by forming each fulcrum into a curved surface corresponding to the outer peripheral surface of the ultrasound probe 100, the fulcrum can be more easily slidable on the outer peripheral surface. Thus, it may be possible to facilitate the removal of the puncture adapter 200.

In general, the puncture adapter 200 is provided with a protruding pinch to reduce the force required for the pinch operation. However, in the intracavitary probe, if the pinch overly protrudes from the ultrasound probe 100, the ultrasound probe 100 is not compatible with the size of the insertion path in a body cavity, which increases the burden on the subject. On the other hand, if the protruding length of the pinch is reduced, more force is required for the pinch operation. When the operation force required for the pinch operation increases, the removal of the puncture adapter 200 becomes difficult.

In this regard, in the puncture adapter 200 of the embodiment, the first operation part 330 is adjacent to the first holding part 321a in the proximal end direction of the plate-like member 310 and is connected to the first holding part 321a. In the same manner, the second operation part 340 is adjacent to the second holding part 321b in the proximal end direction of the plate-like member 310 and is connected to the second holding part 321b. In other words, assuming the first holding part 321a, the second holding part 321b, and a plane passing through a plane on the proximal end side of their connector (310), the first operation part 330 is connected to the first holding part 321a as being adjacent thereto in a direction generally perpendicular to the assumed plane. The second operation part 340 is connected to the second holding part 321b as being adjacent thereto in a direction generally perpendicular to the assumed plane. Besides, if it is defined that a direction that connects the boundary between the first holding part 321a and the second holding part 321b to the outlet of the puncture needle on the distal end side of the puncture adapter 200 (the end in the insertion direction of the puncture needle) is the longitudinal direction of the puncture adapter 200, the first operation part 330 is connected to the first holding part 321a as being adjacent thereto in the longitudinal direction. The second operation part 340 is connected to the second holding part 321b as being adjacent thereto in the longitudinal direction. Note that, in the embodiment, the boundary is an intermediate position of the connector between the first holding part 321a and the second holding part 321b.

With this configuration, the entire puncture adapter 200 can be downsized. Further, less force is required for the pinch operation to remove the puncture adapter 200 from the ultrasound probe 100, which facilitates the removal of the puncture adapter 200.

### [Modification]

In the first embodiment, the puncture adapter is described as being used in an intracavitary probe; however, the embodiment is not limited to this, and may be applicable to an ultrasound probe that is used outside the body cavity. In the case of the probe used outside the body cavity also, if the pinch overly protrudes from the ultrasound probe, this may interfere with the insertion of a puncture needle. Besides, if the pinch overly protrudes from the ultrasound probe, this may interfere with the operation of applying the ultrasound probe to the body surface. In this way, the protruding pinch of the puncture adapter may interfere with a puncture also in the ultrasound probe used outside the body cavity. On the other hand, if the protruding length of the pinch is reduced, more force is required for the pinch operation. As a result, the removal of the puncture adapter becomes difficult.

In this regard, assuming the first holding part, the second holding part, and a plane passing through the connection part of them, also in this modification, one of the operation parts is connected to the first holding part as being adjacent thereto in a direction generally perpendicular to the assumed plane. In addition, the other operation part is connected to the second holding part as being adjacent thereto in a direction generally perpendicular to the assumed plane. In other words, each operation part is adjacent to a holding part (including an engaging part) corresponding thereto, and the direction in which the operation part is adjacent to the holding part is defined as a direction intersecting the gripping direction of the holding parts. Thus, the entire puncture adapter can be downsized. Further, less force is required for the pinch operation to remove the puncture adapter from the ultrasound probe, which facilitates the removal of the puncture adapter.

### Second Embodiment

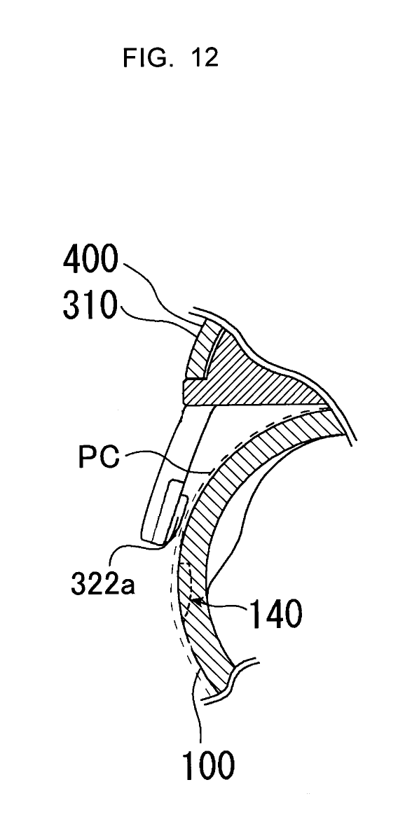

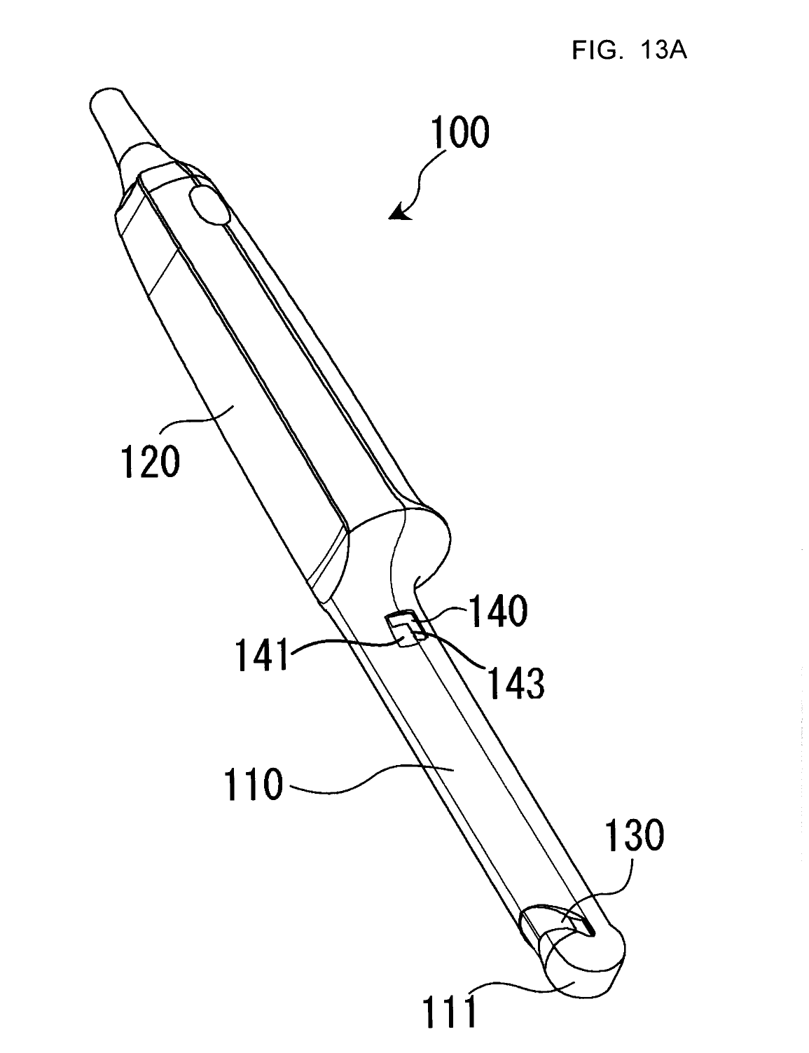

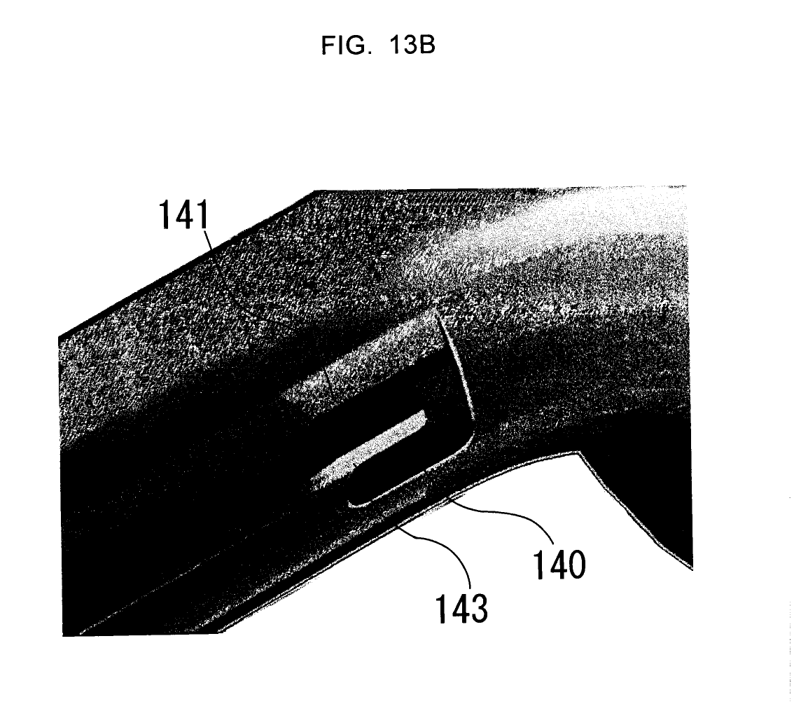

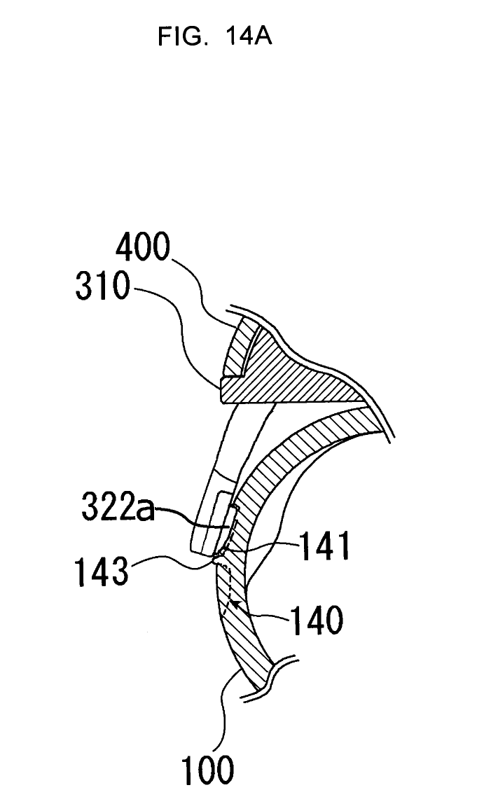

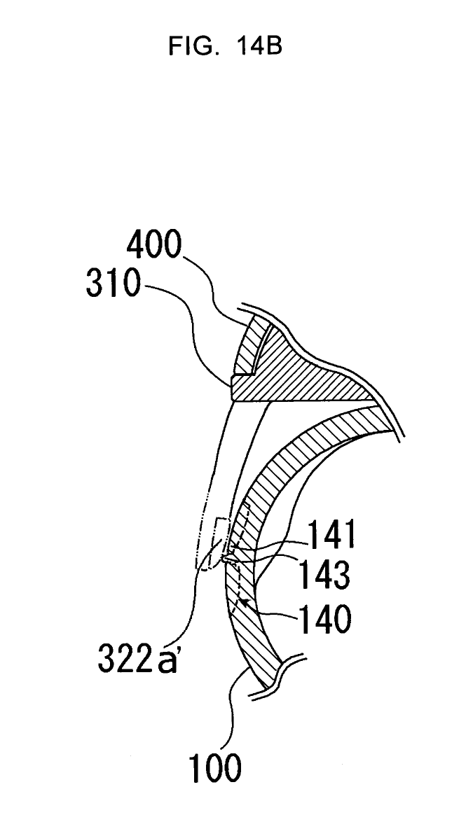

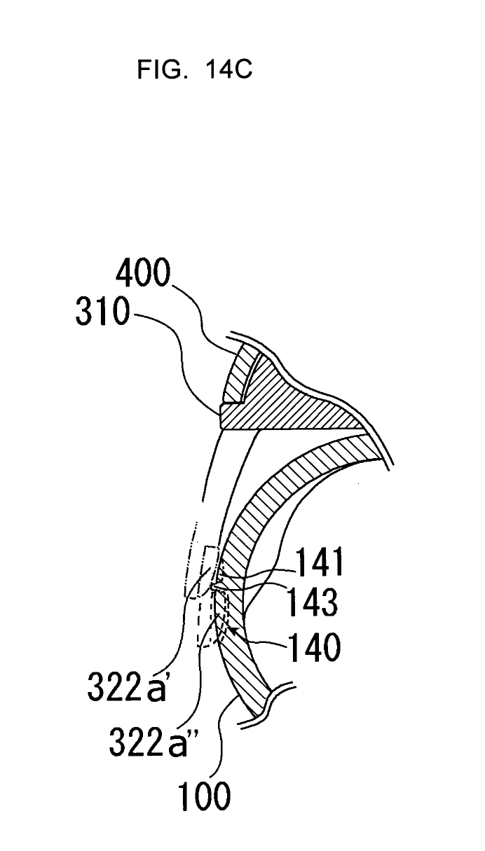

Next, with reference to FIGS. 12 to 14C, a description is given of an ultrasound probe according to a second embodiment. FIG. 12 is a schematic cross-sectional view of a probe cover of the ultrasound probe and engaging parts of the puncture adapter. FIG. 13A is a schematic perspective view of an example of the ultrasound probe 100 of the second embodiment. FIG. 13B is a schematic view of the proximal end side engaging part 140, a sub engaging part 141, and a slope 143 of the ultrasound probe 100 of the second embodiment. FIGS. 14A to 14C are schematic cross-sectional views illustrating the proximal end side engaging part 140 and the like in the ultrasound probe 100 of the second embodiment. FIG. 14A is a schematic cross-sectional view illustrating a state where the engaging part of the puncture adapter engages the sub engaging part 141. FIG. 14B is a schematic cross-sectional view illustrating a state where the engaging part of the puncture adapter runs onto the slope 143. FIG. 14C is a schematic cross-sectional view illustrating a state in which the engaging part of the puncture adapter engages the proximal end side engaging part 140.

In the description of the ultrasound probe 100 of the second embodiment, with respect to the line connecting to the proximal end side engaging parts 140, the position on the side of the puncture adapter 200 to be attached is defined as “upper side” (see FIGS. 14A to 14C). In addition, a direction toward the proximal end side engaging part 140 viewed from the “upper side” is defined as “lower side”. In this assumption, the ultrasound probe 100 of the second embodiment is provided with the slope 143 adjacent to the upper side of the proximal end side engaging part 140. The ultrasound probe 100 is further provided with the sub engaging part 141 adjacent to the upper side of the slope.

As indicated by a broken line in FIG. 12, the ultrasound probe may be fitted with a probe cover. For the intracavitary probe, for example, the cover is made of latex, polyester, or the like. As described above, when the puncture adapter 200 is attached to the ultrasound probe 100, the outer peripheral surface of the ultrasound probe 100 is sandwiched between the first engaging part 322a and the second engaging part 322b. When sandwiching the outer peripheral surface of the ultrasound probe 100, the first engaging part 322a and the second engaging part 322b press the outer peripheral surface of the ultrasound probe 100 by the elastic force of the entire gripper 320 in a direction in which they face each other. Accordingly, the probe cover placed over the ultrasound probe 100 is also pressed against the outer peripheral surface of the ultrasound probe.

When the operator attaches the puncture adapter 200 to the ultrasound probe 100, each of the first engaging part 322a and the second engaging part 322b is slid to the proximal end side engaging parts 140 while pressing a probe cover PC against the outer peripheral surface of the ultrasound probe 100.

In this manner, when the probe cover PC is slid to the proximal end side engaging part 140 as being pressed against the outer peripheral surface of the ultrasound probe 100 by each engaging part, the probe cover PC may be caught in the engaging parts. If the probe cover PC is caught in the engaging part, this increases the resistance to the slide movement of each engaging part of the puncture adapter 200 on the outer peripheral surface of the ultrasound probe 100. If the amount of the probe cover PC caught by the engaging parts increases, the engaging parts may be retained at positions before reaching the proximal end side engaging parts 140 on the outer peripheral surface of the ultrasound probe 100 due to the entrapped probe cover PC.

If this occurs, the operator who is attaching the puncture adapter may mistakenly perceive that the attachment of the puncture adapter 200 to the ultrasound probe 100 is completed. Specifically, despite the fact that the engaging parts are only retained at positions before reaching the proximal end side engaging parts 140 on the outer peripheral surface of the ultrasound probe 100 due to the entrapped probe cover PC, the operator may have a misunderstanding that each engaging part has reached the proximal end side engaging part 140 and is fixed reliably.

To avoid such a situation, as illustrated in FIGS. 13A and 13B, the ultrasound probe 100 of the second embodiment is provided with the sub engaging part 141 and the slope 143 adjacent to the upper side of the proximal end side engaging part 140. Described below are the slope 143 and the sub engaging part 141.

### [Sub Engaging Part]

As illustrated in FIG. 14A, the sub engaging part 141 is arranged adjacent to the upper side of the proximal end side engaging part 140 on the outer peripheral surface of the ultrasound probe 100. The sub engaging part 141 is formed in a recess. In one example, the depth of the sub engaging part 141 is shallower than the depth of the proximal end side engaging part 140.

Incidentally, the proximal end side engaging part 140 corresponds to an example of “first engaged part”. The sub engaging part 141 corresponds to an example of “second engaged part”.

### [Slope]

As illustrated in FIGS. 13B and 14A, the slope 143 is adjacent to the upper side of the proximal end side engaging part 140 on the outer peripheral surface of the ultrasound probe 100, and is adjacent to the lower side of the sub engaging part 141. In other words, the slope 143 is arranged to be interposed between the proximal end side engaging part 140 and the sub engaging part 141. The slope 143 is formed to protrude from the bottom surface of the proximal end side engaging part 140 and the sub engaging part 141 (a surface on the central axis side of the ultrasound probe 100) toward the outside. In the example of FIG. 13B, the slope 143 is formed to have a gentle inclination on the sub engaging part 141 side. Also in this example, the slope 143 is formed to have a steep inclination on the proximal end side engaging part 140 side. The protruding height of the slope 143 coincides with the position of the other outer peripheral surface of the ultrasound probe 100, for example. In other words, the slope 143 is comparable with the other outer peripheral surface of the ultrasound probe 100 in the distance from the central axis.

### [Operation of the Engaging Part of the Puncture Adapter in Attachment Work]

Next, with reference to FIGS. 14A to 14C, a description is given of the operation of the first engaging part 322a and the second engaging part 322b of the puncture adapter 200 with respect to the ultrasound probe 100 of the second embodiment.

### <First Stage>

When the operator presses the puncture adapter 200 against the ultrasound probe 100, first, as illustrated in FIG. 14A, each of the first engaging part 322a and the second engaging part 322b reaches the sub engaging part 141 of the ultrasound probe 100. In this position, the distance between the sub engaging parts 141 is shorter than the distance between the proximal end side engaging parts 140. Therefore, the amount of the entrapped probe cover is small.

### <Second Stage>

When the operator further presses the puncture adapter 200 against the ultrasound probe 100 after the first stage, as illustrated in FIG. 14B, each of the first engaging part 322a and the second engaging part 322b reaches the slope 143. At this time, the first engaging part 322a and the second engaging part 322b that remain in the sub engaging part 141 once run onto the slope 143. Accordingly, the operator who is pressing the puncture adapter 200 feels a resistance.

Besides, as illustrated in FIG. 13B, if the slope 143 is formed to have a gentle inclination on the sub engaging part 141 side, the engaging parts smoothly run onto the slope.

### <Third Stage>

When the operator further presses the puncture adapter 200 against the ultrasound probe 100 after the second stage, as illustrated in FIG. 14C, each of the first engaging part 322a and the second engaging part 322b climbs over the top of the slope 143, and reaches the proximal end side engaging part 140. When the first engaging part 322a and the second engaging part 322b reach the proximal end side engaging parts 140, the engagement is secured. At this time, the first engaging part 322a and the second engaging part 322b that have once run onto the top of the slope 143 (see the part denoted by reference numeral 322a′ in FIG. 14C) slide the proximal end side engaging part 140 side of the slope 143, and reach the proximal end side engaging part 140 (see the part denoted by reference numeral 322a″ in FIG. 14C). Therefore, after feeling a resistance to the pressing operation, the operator feels the engagement between the first engaging part 322a and the like and the proximal end side engaging part 140.

In addition, as illustrated in FIGS. 14A to 14C and the like, if the slope 143 is formed to have a steep inclination on the proximal end side engaging part 140 side, the engaging parts of the puncture adapter side immediately reach the proximal end side engaging part 140 after climbing over the top of the slope 143. Thus, the operator can feel the engagement more certainly.

The ultrasound probe 100 of the second embodiment is provided with the slope 143 adjacent to the upper side (the small diameter side of the outer peripheral surface) of the proximal end side engaging part 140. The ultrasound probe 100 is further provided with the sub engaging part 141 adjacent to the upper side of the slope. Thus, the operator can recognize the feeling of the engagement more certainly. Further, it is possible to avoid a situation where, despite the fact that the engaging parts of the puncture adapter are only retained at positions before reaching the proximal end side engaging parts 140 on the outer peripheral surface of the ultrasound probe 100, the operator mistakenly perceives that the attachment of the puncture adapter 200 is completed.

With the puncture adapter and the ultrasound probe according to the first and the second embodiments described above, the puncture adapter can be easily attached/removed. As a result, the operator can smoothly perform a puncture, and thus it is possible to reduce the burden on the patient.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A puncture adapter comprising a guide of a puncture needle and a gripper configured to grip an ultrasound probe, the puncture adapter configured to be attached to the ultrasound probe by engagement therewith, the puncture adapter further comprising:
a pinch located adjacent to the gripper in an intersecting direction that intersects a direction in which the gripper grips the ultrasound probe, the pinch configured to be used to release a grip of the gripper.

2. The puncture adapter of claim 1, wherein the intersecting direction is a direction that connects the gripper and an end of the guide in an insertion direction in which the puncture needle is inserted.

3. The puncture adapter of claim 1, wherein the pinch is located adjacent to the gripper on a side opposite to an insertion direction in which the puncture needle is inserted.

4. The puncture adapter of claim 2, wherein
the gripper includes a pair of engaging parts configured to engage the ultrasound probe, and
the pinch is located adjacent to the gripper on a side in a direction from the end toward a position between the engaging parts.

5. The puncture adapter of claim 4, further comprising a pair of protrusions protruding toward the ultrasound probe gripped, wherein the protrusions are located closer to the pinch than the engaging parts are.

6. The puncture adapter of claim 5, wherein
the gripper includes a first gripper and a second gripper,
the pinch includes a first operation part and a second operation part,
the protrusions includes a first protrusion corresponding to the first operation part and a second protrusion corresponding to the second operation part,
the first protrusion is located in the first operation part or a boundary between the first operation part and the first gripper, and
the second protrusion is located in the second operation part or a boundary between the second operation part and the second gripper.

7. The puncture adapter of claim 6, wherein the first operation part and the second operation part are configured to make contact with the guide of the puncture needle when brought close to each other to limit movable range of each other.

8. An ultrasound probe comprising:
an ultrasound probe body configured to be inserted in a subject; and
a puncture adapter configured to be attached to the ultrasound probe body to guide a puncture needle, the puncture adapter including
a gripper including a pair of holding parts and a connector configured to connect the holding parts, the gripper configured to hold the ultrasound probe body while gripping part of outer periphery of the ultrasound probe body by the holding parts,
a pair of engaging parts arranged in each of the holding parts, the engaging parts each configured to engage part of the ultrasound probe body, and
a pinch located adjacent to each of the holding parts in a direction that intersects a direction in which the gripper grips the ultrasound probe body, the pinch configured to be used to release a grip of the gripper.

9. An ultrasound probe having a curved outer peripheral surface, comprising:
a pair of first engaged parts arranged spaced apart by a first distance on the outer peripheral surface, the first engaged parts each configured to receive an engaging part of a puncture adapter;
a pair of second engaged parts arranged spaced apart by a second distance that is shorter than the first distance on the outer peripheral surface, the second engaged parts each configured to receive an engaging part of the puncture adapter;
a first slope formed between one of the first engaged parts and corresponding one of the second engaged parts; and
a second slope formed between another of the first engaged parts and corresponding one of the second engaged parts.

10. The ultrasound probe of claim 9, wherein the first slope and the second slope each have a gentle inclination on a side of the second engaged parts.
